(19) United States
(12) Patent Application Publication
(10) Pub. No.: US 2011/0295054 A1
(43) Pub. Date: Dec. 1, 2011

(54) Method of Filling an Intraluminal Reservoir with a Therapeutic Substance

(76) Inventors: Jeffrey L. Aldridge, Lebanon, OH (US); Gregory J. Bakos, Mason, OH (US); Sean P. Conlon, Loveland, OH (US); Michael S. Cropper, Edgewood, KY (US); Denzel Z. Herrera-Davis, Cincinnati, OH (US); Daniel F. Dlugos, JR., Middletown, OH (US); Lucas B. Elmer, Cincinnati, OH (US); Jason L. Harris, Mason, OH (US); Christopher J. Hess, Cincinnati, OH (US); Jeffrey D. Messerly, Cincinnati, OH (US); Mark S. Ortiz, Milford, OH (US); Mark D. Overmyer, Cincinnati, OH (US); Alessandro Pastorelli, Roma (IT); Michael J. Stokes, Cincinnati, OH (US); Foster B. Stulen, Mason, OH (US); Suzanne Thompson, West Chester, OH (US); Richard W. Timm, Cincinnati, OH (US); James W. Voegele, Cincinnati, OH (US); Lauren S. Weaner, Cincinnati, OH (US); William B. Weisenburgh, II, Maineville, OH (US); Tamara S. Vetro Widenhouse, Clarksville, OH (US); James A. Woodard, JR., Mason, OH (US); Mark S. Zeiner, Mason, OH (US); Andrew M. Zwolinski, Hamburg (DE)

(21) Appl. No.: 13/104,095

(22) Filed: May 10, 2011

Related U.S. Application Data
(60) Provisional application No. 61/348,264, filed on May 26, 2010.

(51) Int. Cl. A61F 2/04; A61M 31/00; A61M 25/00

(52) U.S. Cl. 600/37; 604/500; 604/516

(57) ABSTRACT

Methods described herein involve introducing a nasogastric tube into a patient, connecting the nasogastric tube with a reservoir, anchoring the nasogastric tube with the nasal cavity, and introducing a substance into the reservoir through the nasogastric tube.

### PRIORITY

This application claims priority to U.S. Provisional Patent Application Ser. No. 61/348,264, entitled “Method of Filling an Intraluminal Reservoir with a Therapeutic Substance,” filed May 26, 2010, the disclosure of which is incorporated by reference herein.

### BACKGROUND OF THE INVENTION

This invention relates to systems and methods for initiating feelings of satiation. In particular, this invention relates to systems and methods for using intestinal brake for the control of hunger.

Several decades of medical research have produced advances in the treatment of obesity through bariatric surgery. The use of intestinal bypass been surpassed by the gastric bypass surgery and also by the use of gastric bands. However, complications from such surgical procedures are frequent.

It is known that certain gastrointestinal hormones influence satiation and satiety. Hormones such as Glucagon-like peptide-1 (GLP-1) and similar analogs are the basis of the oral appetite suppressants. However, weight loss with prescribed hormones and orally administered drug analogs is limited due to timing, patient compliance, etc. Additionally patients develop tolerances and become refractory to the drugs. Therefore there remains a need in the art for improved systems and methods for the control of hunger in a patient using hormonal regulators.

### BRIEF DESCRIPTION OF THE DRAWINGS

FIG. 1A is a side view of a saphenous vein.

FIG. 1B is a side view of a saphenous view.

FIG. 1C is a side view of a reduced diameter endogenous vessel.

FIG. 2 is a side view of a transfer system having a one-way valve.

FIGS. 3A-D are side views of a transfer system.

FIG. 4 is a perspective view of a gastric band system.

FIG. 5A is a side view of a gastric band system.

FIG. 5B is a side view of a gastric band system.

FIG. 6A is a side view of a biomorph system.

FIG. 6B is a side view of a biomorph system.

FIG. 7A is a side view of a biomorph system.

FIG. 7B is a side view of a biomorph system.

FIG. 8A is a side view of a biomorph system.

FIG. 8B is a side view of a biomorph system.

FIG. 9A is a side view of a biomorph system.

FIG. 9B is a side view of a biomorph system.

FIG. 10 is a perspective view of MEMs system.

FIG. 11A is a side view of a tube system intraluminally placed in a bowel.

FIG. 11B is a side view of a tube system intraluminally placed in a bowel.

FIG. 12A is a side view of a reservoir system.

FIG. 12B is a side view of a reservoir system.

FIG. 12C is a side view of a reservoir system.

FIG. 13 is a side view of an Alzet pill.

FIG. 14 is a side view of an implanted nasal fill port system.

FIG. 15A is a side view of an implanted nasal fill port system.

FIG. 15B is a side view of an implanted nasal fill port system.

FIG. 15C is a side view of an implanted nasal fill port system.

FIG. 15D is a side view of an implanted nasal fill port system.

FIG. 15E is a side view of an implanted nasal fill port system.

FIG. 15F is a side view of an implanted nasal fill port system.

FIGS. 16A and 16B are side views of a reservoir system.

FIG. 17A is a side view of a therapeutic delivery system.

FIG. 17B is a side view of a therapeutic delivery system.

FIG. 17C is a side view of a therapeutic delivery system.

FIG. 17D is a side view of a microneedle delivery system.

FIG. 17E is a side view of a microneedle delivery system.

FIG. 17F is a side view of a microneedle delivery system.

FIG. 17G is a side view of a microneedle delivery system.

FIG. 18A is a side view of a piezoelectric system.

FIG. 18B is a side view of a piezoelectric system.

FIG. 18C is a side view of a piezoelectric system.

FIG. 19 is a perspective view of a delivery system.

FIGS. 20A and 20B are perspective views of a delivery system.

FIGS. 21A and 21B are perspective views of a delivery system.

FIG. 22 is a perspective view of a delivery system.

FIG. 23 is a perspective view of a delivery system.

FIG. 24 is a graph of intra-band pressure.

FIG. 25 is a perspective view of a delivery system.

FIGS. 26A and 26B are perspective views of a delivery system.

FIG. 26C is a perspective view of a delivery system.

FIG. 27 is a perspective view of an electrical probe.

FIG. 28 is a side view of a therapeutic delivery system.

FIG. 29 is a side view of a therapeutic delivery system.

FIGS. 30A and 30B are perspectives view of a therapeutic delivery system.

FIG. 31 is a side view of a band delivery system.

FIG. 32 is a side view of a band delivery system.

FIG. 33 is a schematic view of a therapeutic delivery system.

FIG. 34 is a perspective view of a therapeutic delivery system.

FIG. 35 is a perspective view of a therapeutic delivery system.

### DETAILED DESCRIPTION

The following detailed description is intended to be representative only and not limiting as to devices and methods for delivering a therapeutic substance to induce intestinal brake. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the invention as presented herein.

In a first embodiment of a transfer system, a catheter tube is connected to a reservoir with a delivery interface. The catheter may extend directly from a connection at the reservoir to the delivery interface. Alternatively, the catheter may weave throughout the abdomen. The catheter may also be fixed to internal tissue sutures or equivalent fixation means. The catheter can be rigid, flexible, and of varying cross-sectional shapes. Multiple catheters may also be used to transfer fluid to additional sites.

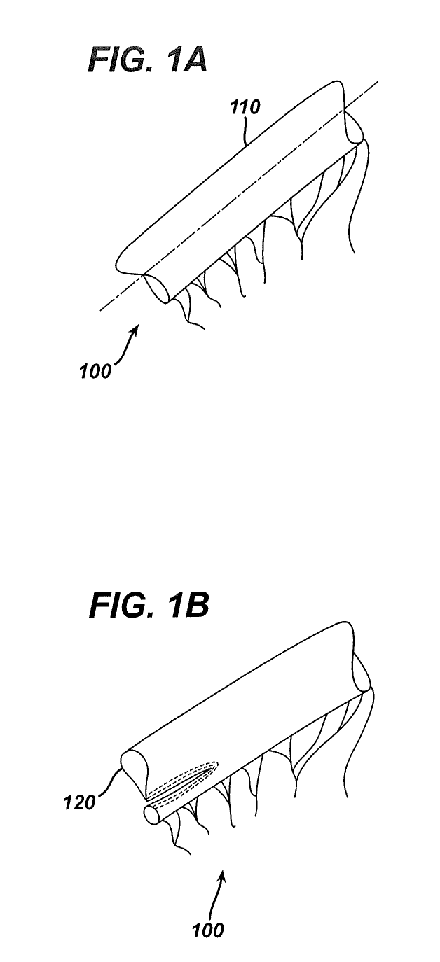

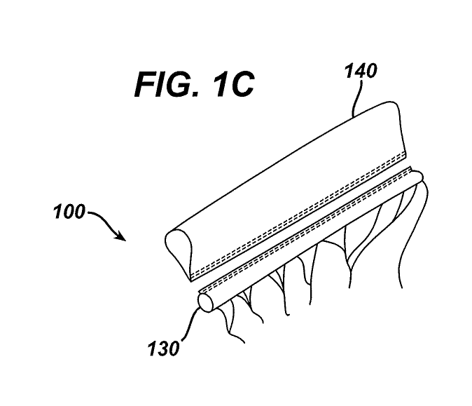

Turning now to FIGS. 1A-C, the transfer system 100 is comprised of a harvested portion of the body such as the saphenous vein 110 or a diameter reduced portion of the gastrointestinal tract 120 which is left attached to the mesentery to enhance biocompatibility of the greater part of the system. The vessel 110, 120 receives transfer of therapeutic agents via a catheter. In addition, the vessel 110, 120 may participate in signalling since it remains vascularized and contains some of the cells which cause signalling to occur (e.g., L-cells). In an embodiment of FIG. 1C, a reduced diameter endogenous vessel 130 is shown separated from an original lumen portion 140, which is discarded.

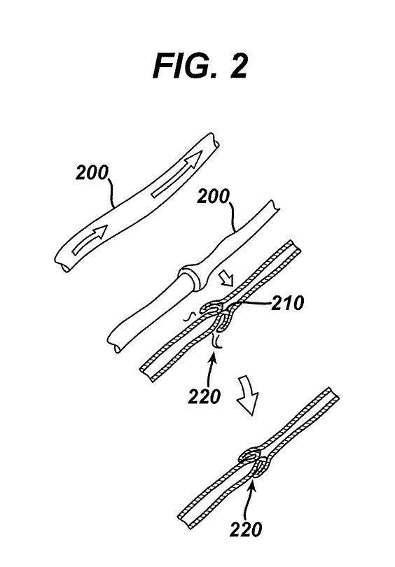

In another embodiment of the present invention as seen in FIG. 2, a catheter 200 is outfitted with a one-way valve 210 and may be used to ensure transfer of therapeutic substances in a desired direction and prevent infusion of unwanted fluid or particulate. Several one-way valves may be used including as examples one-way duckbill valves or flapper valves. In the case of a biologic catheter such as is made from endogenous material, a valve could be formed by permanently reshaping tissue using an intussuception. The portion of lumen that is circumferentially folded over on itself is then stitched at various points 220 circumferentially to cause serosa-to-serosa healing for permanence of the valve 210.

In another embodiment of a transfer system, a head loss coil is used. With a head loss coil (not shown), a long coil of tubing with a small diameter-to-length ratio is used to connect a pressurized reservoir to the delivery interface. This connection permits the high-pressure therapeutic substance in the reservoir to be converted to a lower pressure fluid at the delivery site. This permits a steady and slow drip of therapeutic substances to be maintained for an extended period of time.

In yet another embodiment, a catheter with passive flow control is presented. A catheter with inline flow control is used to insure a preset and constant dosage (volume flow) of therapeutic substance delivery. By using this device, fluid forced to flow from a variable, pressurized reservoir is converted into a constant and known volume flow independent of changing reservoir pressure. An example of a device that achieves this result is the Chronoflow device by Debiotech of Lausanne, Switzerland. The device provides a solution for converting fluid at variable pressure into a constant rate flow without the need for power input or a control system.

Another embodiment of the transfer subsystem is a catheter with an inline pump. The transfer of therapeutic substances from the reservoir to the small bowel is driven by the pump.

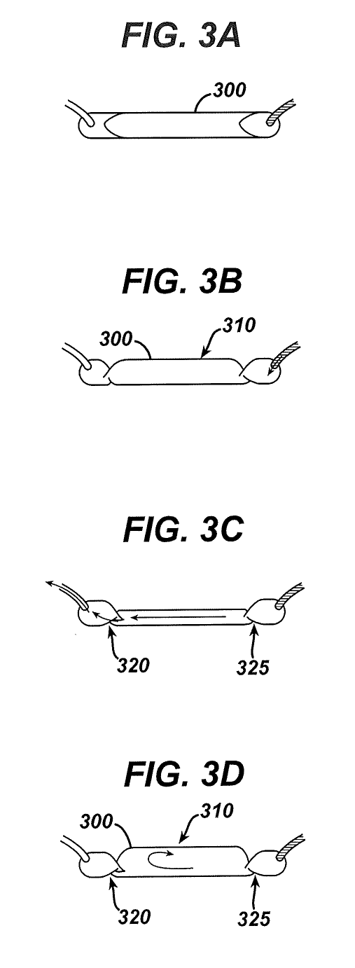

In examples of pumps for use with the transfer system as seen in FIGS. 3A-3D, a peristalsis driven pump is used with a gastric band 300. The pump includes a pumping section 310 in the gastric band 300 composed of valves 320, 325 that are nested between more elastic portions of the band 300 (e.g. fluid reservoirs) and outer, less elastic portions of the band 300 used for holding the band 300 together. In the embodiment as shown in FIG. 3, pressure exerted by passing bolus of food causes a rise in the pressure of the gastric band 300. The increased pressure forces delivery of the therapeutic substance. The one-way valves insure flow in the desired direction.

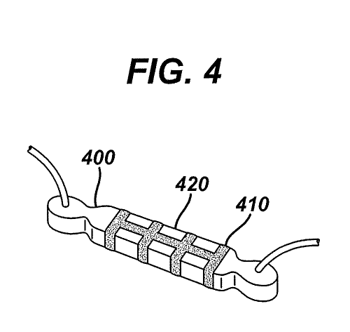

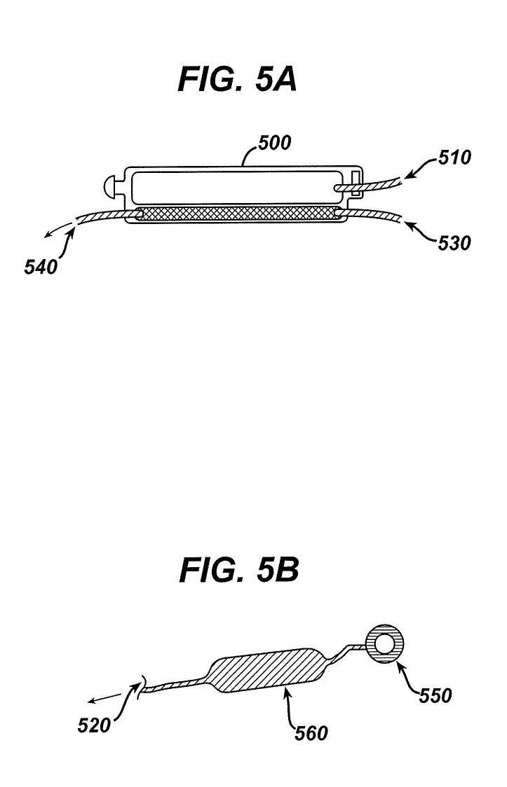

In FIG. 4, another embodiment of a gastric band 400 uses a collapsible, flexible frame 410. The collapsible frame 410 insures that the pumping section 420 of the band 400 opens completely after each compression. The frame 410 therefore insures that a consistent and efficient pumping action is maintained throughout operation of the band 400. FIGS. 5A and 5B show another embodiment using a peristalsis driven gastric band pump. In the first example of FIG. 5A, a gastric band 500 is shown having a band inlet 510. The band 500 is operatively coupled to a pump section 520 having an inlet 530 and an outlet 540. Operation of the pump section 520 is controlled by flow from inlet 530 to outlet 540. In FIG. 5B, the pump section is shown having a velocity port 550 and a fluid reservoir 560 to regulate the required pumping force. Generally, the pumping section 520 of the band 500 is separated from the normal, restrictive section of the band 500. By separating the two functions, the band 500 maintains its original function as a gastric restriction device, but has the added ability to pump therapeutic substances as needed.

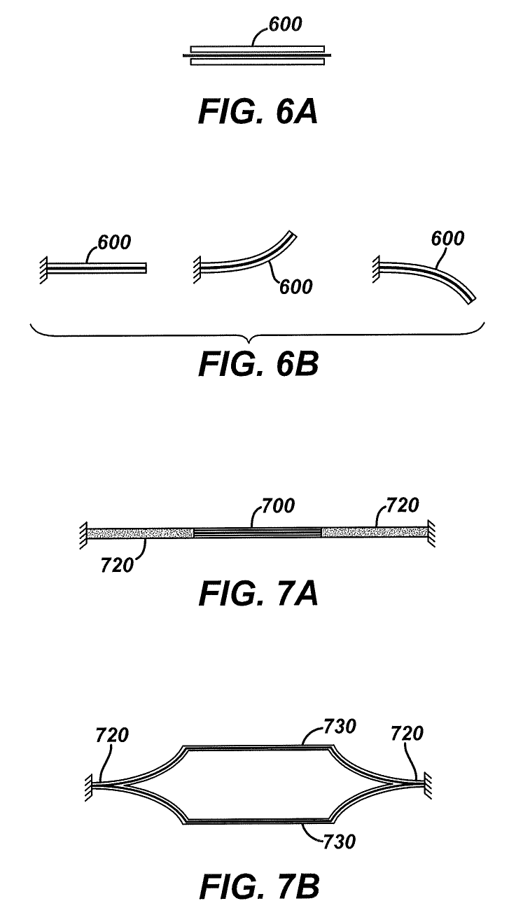

In other embodiments, piezoelectric cantilevers as shown in FIGS. 6A-D are used with the pumps. Piezoelectric cantilevers 600 are smart devices, in that when a voltage is applied across the piezoelectric material, the cantilever is forced to bend. While a positive voltage causes bending in a first direction, a negative voltage causes movement in the opposite direction. Cantilevers 600 are constructed in a unimorph form where one piezoelectric plate is bonded to a shim or a bimorph form where piezoelectric plates are attached to both sides of a center shim. The advantage of bimorph and unimorph cantilevers 600 is that they achieve much greater displacements compared with monolithic piezoelectric plates or disks. Generally “soft” piezoelectric (e.g., PZT-5) materials are used. Shim materials are metals or plastics. Plastic shims are common to piezoelectric fans. Metal shims are more rigid and are common for actuators. Unimorph cantilever construction is typical in small MEMS devices used for sensing (e.g., artificial noses). In this case, zinc oxide deposited on one side of the cantilever forms the piezoelectric materials. Other lead-free piezoelectric materials may include barium titanate.

In FIGS. 7A and 7B, four bimorphs 720 are connected in pairs and joined at the ends of the cantilever 700 with or without additional connecting members 730. When no voltage is applied, the device is flat with no appreciable internal area or volume, as shown in FIG. 7A. When the cantilevers 700 are actuated with a single polarity, they all flex away from the center, creating an internal area or volume, as shown in FIG. 7B. Exterior ends are rigid earthed walls. The connector members 730 may be elastomeric to accommodate stretching when activated. The connector may be rigid.

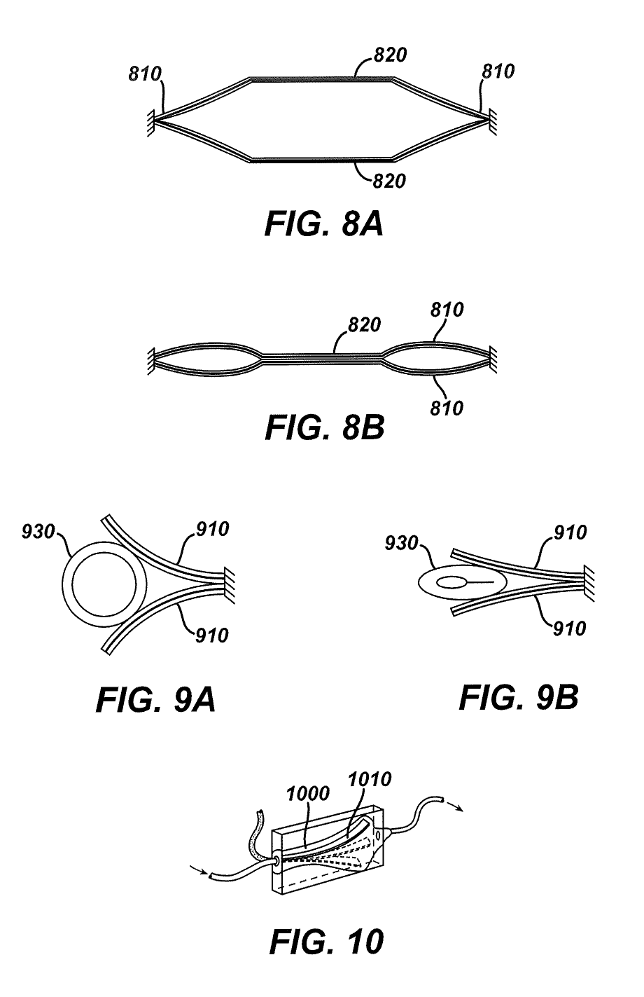

In another embodiment of FIGS. 8A and 8B, two pairs of bimorphs 810 are mounted at a predetermined angle such that a first pair is positioned between the other biomorph pair (e.g., 90 degrees). The two pairs of biomorphs 810 are then joined at their ends to one of the biomorphs of the other pair. They may be joined with or without one or more connector members 820, and thereby define an internal area/volume. When the cantilevers are flexed inward, the area or volume decreases (ideally to zero). This configuration therefore uses four biomorphs wired in parallel and in a normally open configuration. If desirable, the bimorphs 810 may be actively flexed outward by reversing the polarity to increase the pumping volume, as shown in FIG. 8B.

In yet another embodiment of FIGS. 9A and 9B, a pair of bimorphs 910 squeezes the material, tissue, vessel, bowel, reservoir, or pouch 930 that is placed between the two bimorphs 910. In still another embodiment, an alternative series of peristaltic pumping systems is used along a length of bowel (not shown). As one pumping system is opening, another pumping system is closing to create peristaltic pumping. Proper selection of polarities would allow both devices to be driven in parallel. However, phase control should be considered. Any number of pairs could be placed along the bowel in a contiguous chain or separate sites. This embodiment would use one pump similar to that described in FIGS. 7A and 7B, and alternate with a pump similar to that described in FIGS. 8A and 8B, and so forth.

In still another embodiment, a MEMS artificial nose sensor is configured as a microfluidic pump by a MEMS array of cantilevers (not shown) driven as a microfluidic pump. The pump may be combined with the sensor to introduce fluids with target components or to “backwash” the sensor to reset it. And in yet another embodiment of FIG. 10, a single or array of cantilevered beams are encased in a housing 1000. An alternating current is applied to the beams to flex up and down rapidly by means of a piezoelectric functionality. The internal chamber of the housing 1000 is shaped such that it forms the extreme flexed shape of the beam 1010. The beam 1010 acts as a flapping pump forcing fluid from the inlet of the casing to the outlet.

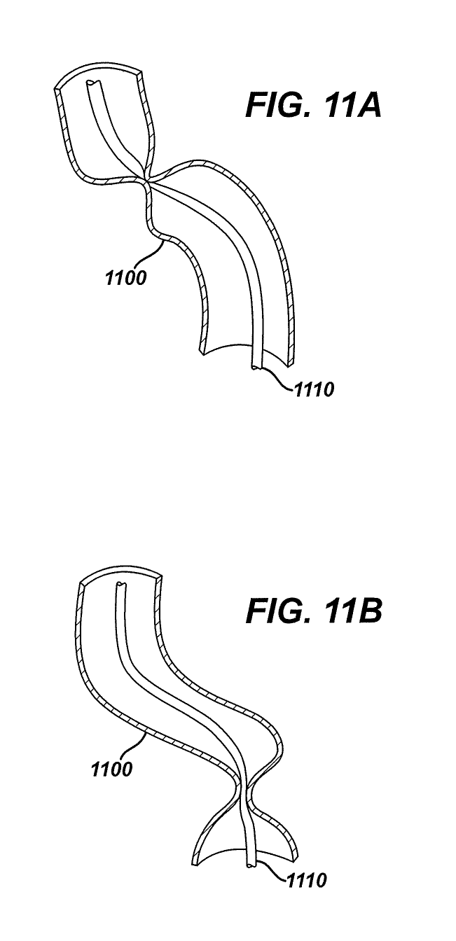

Turning now to FIGS. 11A and 11B, a flexible tube 1110 is placed intraluminally in the small bowel 1100. A peristaltic wave may act to squeeze therapeutic substances distally in the tube as shown in FIGS. 11A and 11B. In another embodiment, a catheter is used such that peristalsis of the lumen accelerates the contents of the catheter. In such an embodiment, the catheter is comprised of a series of funnels with one-way valve outlets. As a volume of content is introduced into the funnel, the peristaltic wave squeezes the contents through the relatively small outlet with the one-way valve into the next chamber. The nozzle affect accelerates the flow of nutrient to the next chamber. The size and shape of the funnel segments will affect the acceleration of the content.

An electroactive polymer peristaltic pump may be used with the aforementioned embodiments since the contractions of the individual segments force the fluid along the path. One type of electoactive polymer-based pump is shown in commonly assigned U.S. Pat. No. 7,353,747 to Swayze et al. In addition, the position of the reservoir subsystem and the delivery interface subsystem may be optimized to make the use of a transfer system unnecessary. In some embodiments, the reservoir is refillable through minimally invasive means or does not require refilling.

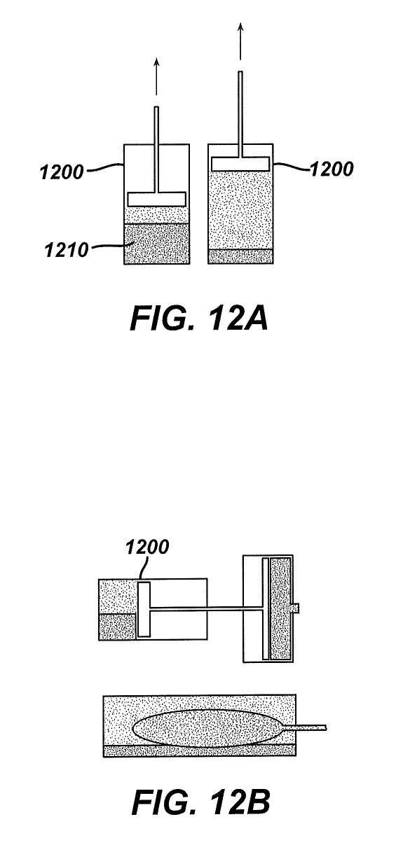

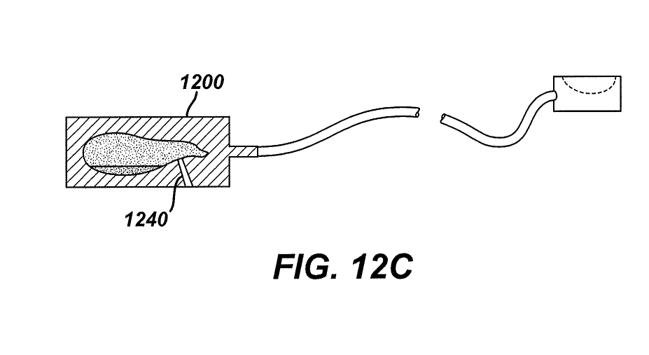

In a first example of the reservoir subsystem and as seen in FIGS. 12A-12C, a substance 1210 with a low saturation pressure is used to maintain a rigid reservoir 1200 at a semi-constant pressure even after delivering a large percentage of the reservoir contents. By maintaining a constant pressure, constant flow rate is achieved. Candidate substances include R-134a.

In all of the aforementioned examples, the reservoirs of both the constant pressure fluid and the substance being delivered may be refilled by a catheter 1240. The catheter connects with a fill port including a self-sealing silicon septum. The delivery rate of the therapeutic is adjustable by controlling the amount of fluid in the pressurizing reservoir.

Existing reservoirs used for implanted insulin delivery systems are a proven technology adaptable for the delivery of therapeutic substances as described with respect to the aforementioned systems. Pumps such as the Debiotech MEMS pump may be used to deliver the therapeutic from a reservoir, powered by a rechargeable implantable battery. The battery in some cases may be rechargeable by inductive coupling. The reservoir from which the pump draws the therapeutic substance may be in a bladder supplied via catheter from a subcutaneous fill port such as is used in an adjustable gastric band velocity port.

One embodiment of a reservoir uses a gastric band. Instead of using the typical pressurization fluids (e.g. saline), the band is filled with a therapeutic substance. The reservoir may include the existing fill port. Added connections would connect the band to the transfer system, which would then connect to the delivery interface.

In one example, the band uses a highly flexible, low perfusion bladder internally to prevent unintentional perfusion of the fluid from within the band to the area outside the band. The use of silicone is known to be susceptible to such perfusion issues. Alternatively, the bladder is attached and situated alongside the band. The bladder is optionally operatively coupled to the system for the restriction component of the band. During implantation, the band is either installed in the customary position to achieve the additional banding effect on satiety, or the band is installed in a location that does not have a direct impact on weight. A subcutaneous fill port may be used to refill the therapeutic substance in the reservoir. Also, the presence of a fill port may eliminate the need for a reservoir altogether.

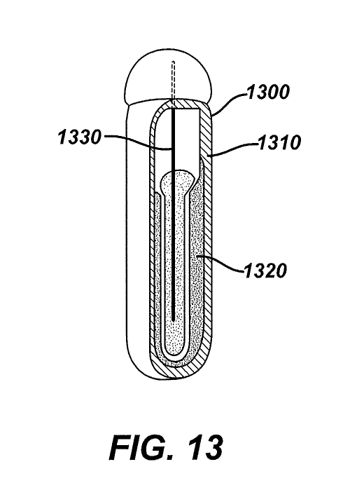

Alternatively as seen in FIG. 13, the reservoir may be an Alzet pill 1300 that absorbs water from the surrounding environment by osmosis through a semi-permeable membrane 1310 causing swelling of an osmotic layer 1320. This swelling in turn forces the therapeutic in the inner chamber to be expelled at a constant rate over time through a flow moderator 1330.

In yet another embodiment, a nasal fill port is used to fill the reservoir. Use of a nasal fill port offers certain advantages such as decreased invasiveness in comparison to subcutaneous fill ports and the use of intraluminal methods that do not require a fluid path to be transluminal. Embodiments of nasal fill ports are presented.

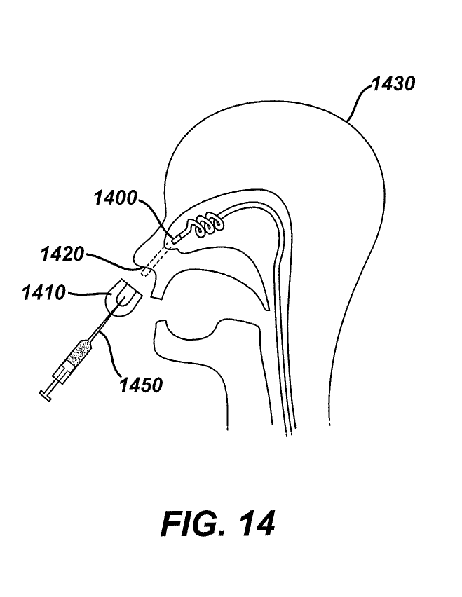

In a first embodiment, as seen in FIG. 14, a medical professional will access a nasal fill port 1400 by placing a magnet 1410 close to a nostril 1420 of a patient 1430. A magnetically attractive ring is positioned around the proximal tip of the tube and may be drawn toward the magnet 1410 to allow a needle 1450 to be refilled via a self-sealing septum. By coiling a catheter, a natural spring is created that retracts the tube into the nasal cavity when it is not being refilled.

In another embodiment, a catheter having a distal tip filled with an elastomer is mounted into the sinus close enough to the nostril to allow access to the fill port while remaining out of view. During filling, a medical professional accesses the port, inserts a needle through the elastomer and into the catheter, and injects the therapeutic substance. Afterwards, the professional recaps the catheter.

In still another embodiment, a catheter with a stopper is mounted in the sinus deep enough to be non-visible, yet close enough to the nostril to permit access to the fill port. During filling, a medical professional accesses the port, uncaps a catheter, injects the therapeutic substance, and recaps the catheter.

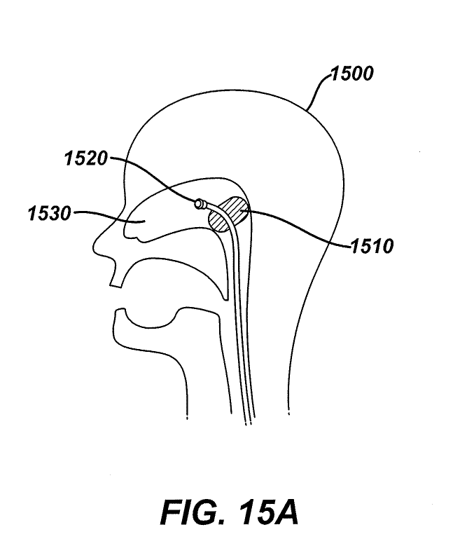

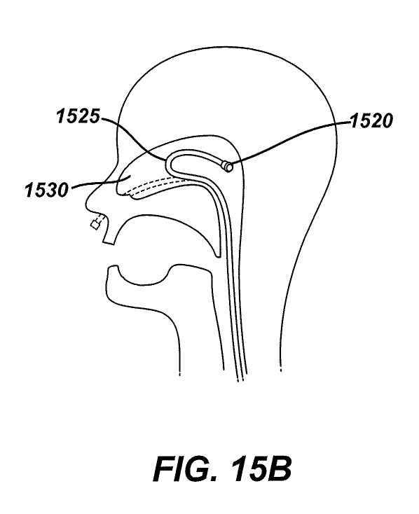

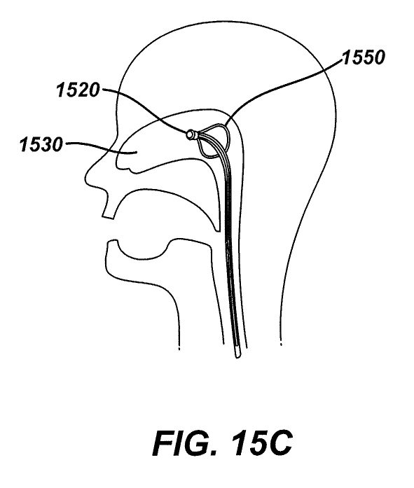

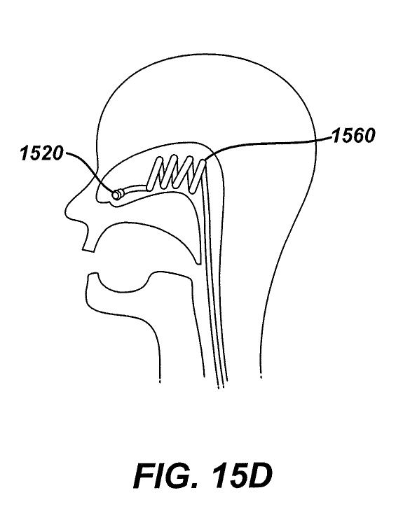

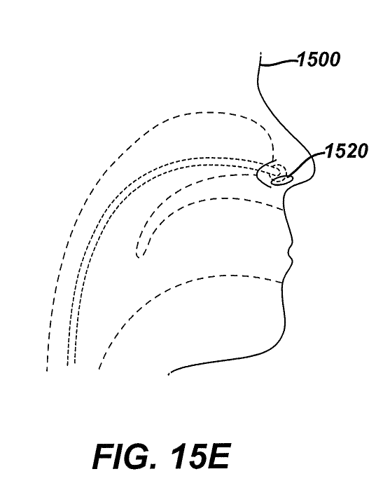

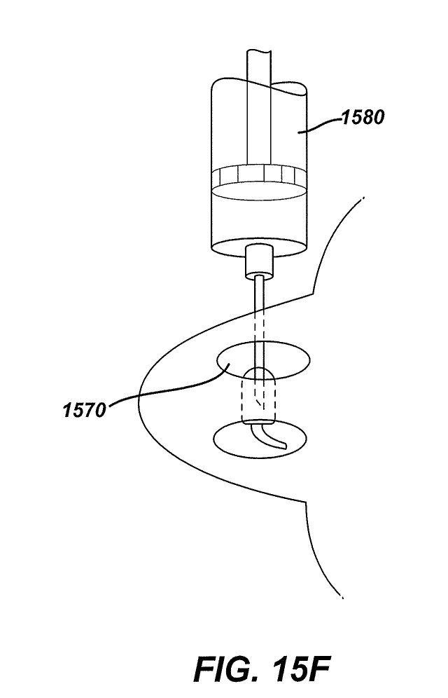

In still other embodiments are shown with respect to FIGS. 15A-15F. In FIG. 15A, an inflatable balloon 1510 anchors a fill port 1520 in a nasal cavity 1530 of patient 1500. During installation, the medical professional inflates the balloon 1510. To refill the system, the professional inserts a needle through the nostril of the patient 1500 and into the port 1520. Scopes may be used to visualize the process.

In another embodiment of a fill port shown in FIG. 15B, a fill port 1520 has an inherent bend in the catheter 1525 near a proximal end. The device functions such that during waking hours, the bend in the catheter 1525 anchors the system in the nasal cavity 1530. During filling, the tube is straightened and extracted from the nose for access to the port 1520. After refilling, the port 1520 is re-bent and placed back in the nasal cavity 1530.

In still another embodiment of a fill port shown in FIG. 15C, flexible arms 1550 anchor the port 1520 in the nasal cavity 1530. During installation, the flexible arms 1550 are compressed, the port 1520 is inserted into the nasal cavity 1530, and the arms 1550 are allowed to expand. To refill the system, a medical professional inserts a needle through the nostril and into the port 1520. A scope may be used to visualize the process. FIG. 15D teaches a similar design using a port 1520 having a coiled anchor 1560. In FIG. 15E, an example is presented wherein the fill port 1520 is implanted in the soft tissue of the septum of a patient 1500 such that the port 1520 is readily visible. The resulting implanted port 1520 is easily accessible and invisible to plain view. The color of the fill port 1520 may also be adjusted to assist in making the port 1520 invisible to plain view. FIG. 15F shows the fill port 1520 being refilled. In the figure, the nostrils 1570 are pushed back and a needle 1580 is inserted into the port. An elastomeric plug may be used to facilitate multiple fills.

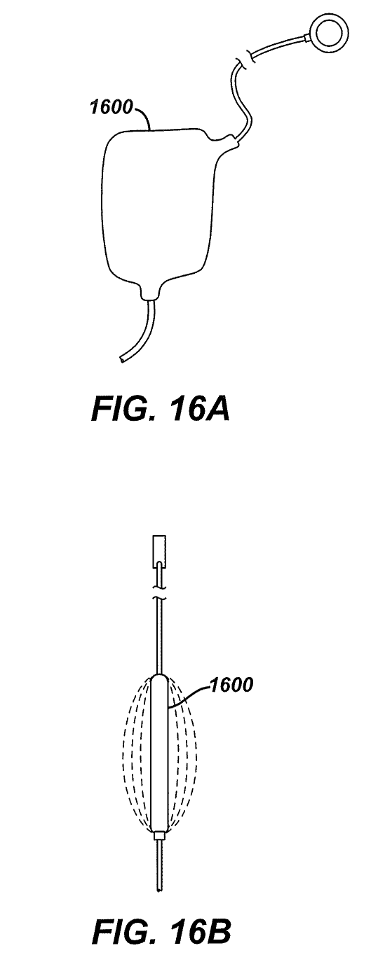

Turning to FIGS. 16A and 16B, another embodiment of a reservoir 1600 is shown where the reservoir 1600 itself would expand due to the pressure of its contents. In this way, the reservoir 1600 itself would exert a range of pressure on its contents, forcing delivery. In one example, the reservoir 1600 is constructed of a biocompatible elastic polymer, and incorporates a band type subcutaneous fill port or a nasal fill port. In other examples, the reservoir is also elastic and includes mechanical hard stops to limit the expansion range.

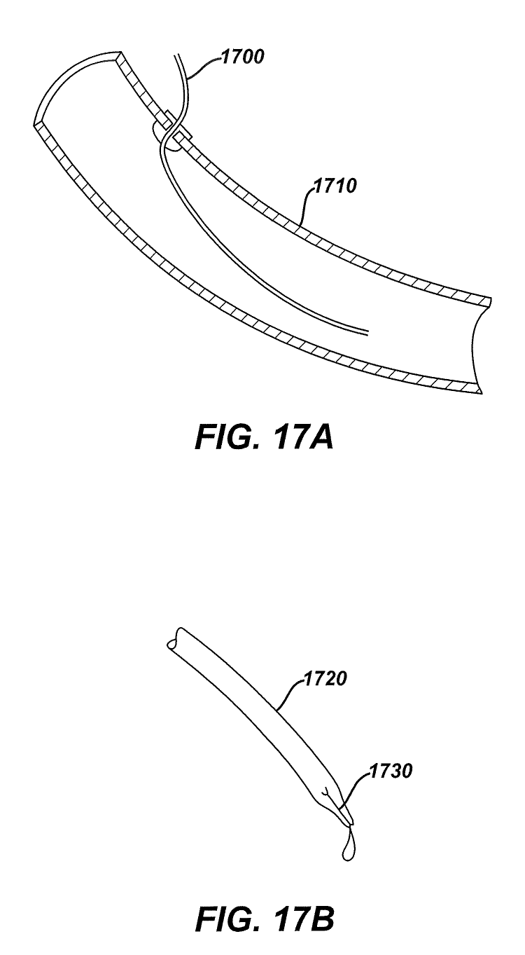

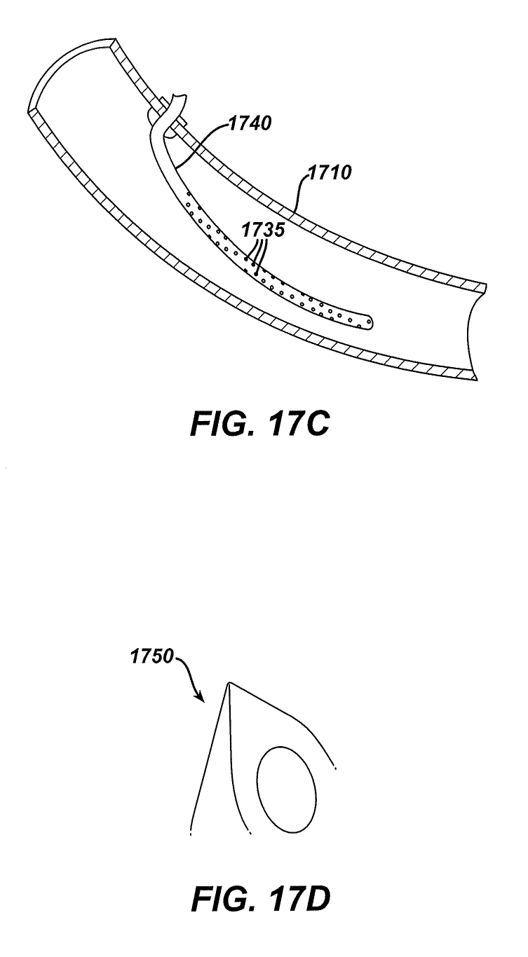

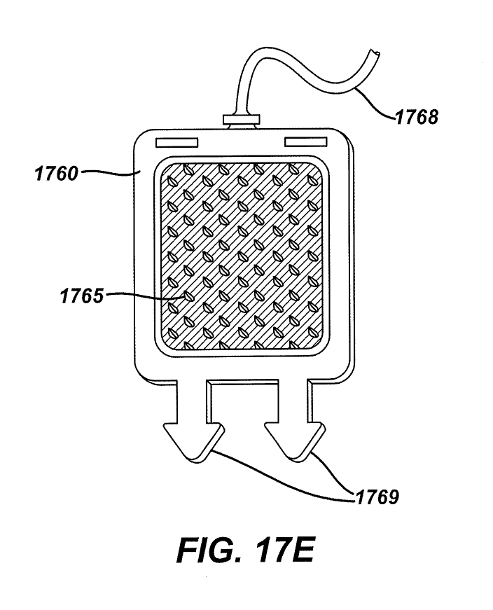

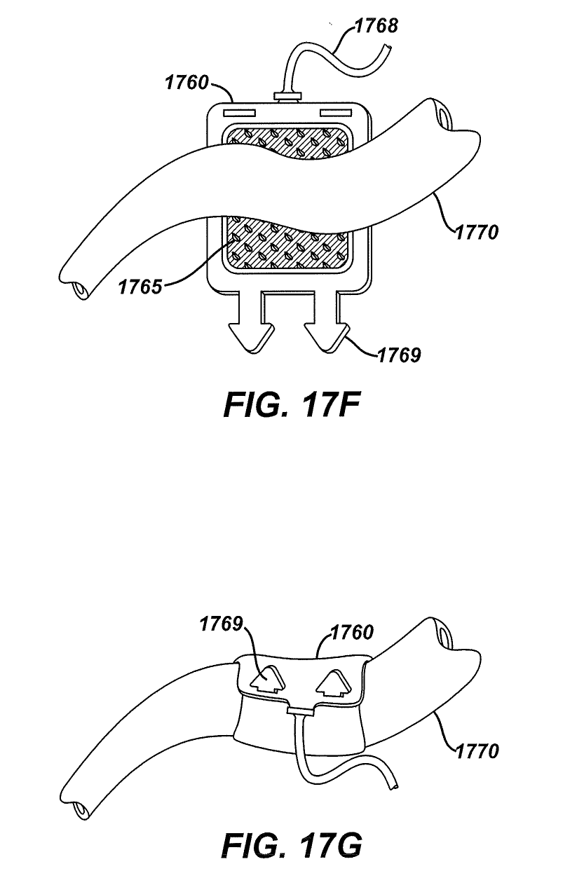

The delivery interface subsystem insures delivery of the therapeutic substance to the desired location (e.g. the small bowel). Embodiments of the delivery interface are hereafter presented. In a first embodiment of FIG. 17A, the delivery interface is a simple open tube 1700. The tube 1700 passes through the lumen 1710 to deliver the therapeutic substance. Alternatively, the delivery interface could enter the biliary tree trans-hepatically, where it could then extend into the lumen of the small bowel. In FIG. 17B, another embodiment of a delivery interface is a catheter 1720 with one-way duck billed valve 1730. The valve 1730 permits therapeutic substances to exit the system easily while preventing intestinal matter from entering the system and inhibiting the formation of unwanted clogs. In yet another embodiment, a delivery interface includes a fine mesh to prevent particulate from entering the system while permitting therapeutic substance transfer. And in still another embodiment, a delivery interface includes some amount of radiopaque material for location of the delivery interface through imaging technology means.

As seen in FIG. 17C, a delivery system embodiment includes a perforated tube 1740 having holes 1735. The perforated tube 1740 spreads out delivery of therapeutic substance for a selected length of small bowel, and only may only include one site of luminal transection. In another delivery system embodiment, microneedle technology is used as seen with respect to FIGS. 17D and 17E. Microneedle technology 1750 provides a painless method of creating a bridge through the tissue barrier both for a wide range of drug therapies and also for extracting diagnostic information. A flexible patch 1760 is outfitted with a microneedle array 1765 to deliver a therapeutic substance through the lumen of the bowel at a desired delivery point. The depth of penetration of the microneedles may be selected such that they penetrate only the first external layer of the bowel. Alternatively, the depth of penetration may include two or more layers of bowel tissue. The patch may include a fluid connection 1768 for regulation of fluid flow and latches 1769 for mechanical positioning.

As seen in FIG. 17F, a microneedle array is attached to a flexible backing. The backing is hollow such that a tube can connect it with the overall therapeutic substance delivery system. The device is then installed adjacent to a desired delivery site and wrapped around a portion of a bowel 1770 as seen in FIG. 17G. Suturing and similar methods are used to prevent further relative movement of the device and bowel. The patch 1760 may be outfitted with the pump described below to insure the delivery of therapeutic substance over the microneedle interface.

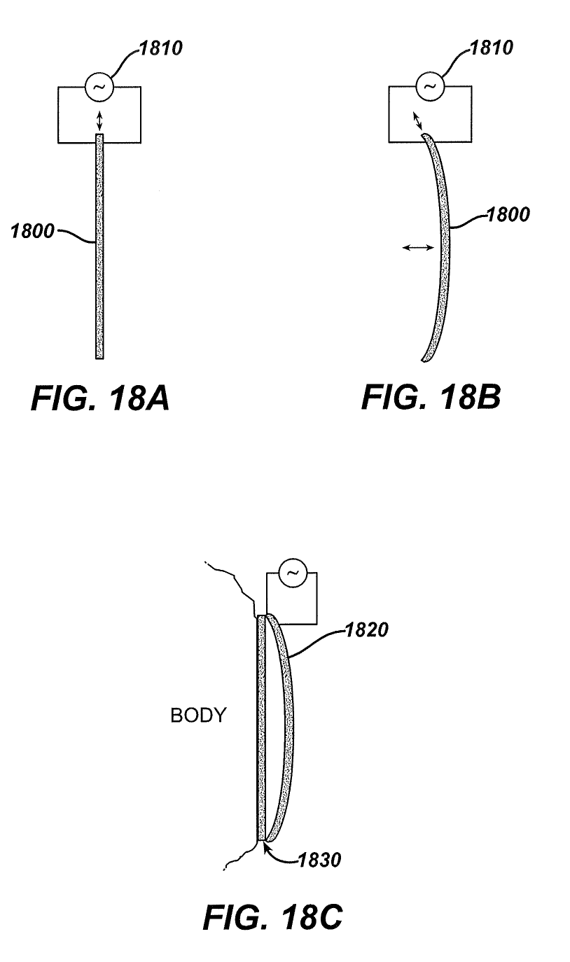

The invention uses common piezoelectric polymer film PVDF sheet as seen with respect to FIG. 18A. When an alternating voltage 1810 is applied across the thickness of the PVDF sheet 1800, the sheet 1800 is caused to expand and contract along its length in one or more directions. This expansion, as shown in FIG. 18B, results in a pumping action. When the PVDF is held slightly curved, it then pumps normal to the surface. A simple illustration of this concept is performed by attaching a PVDF sheet as a speaker to a radio or other electronic sound producing device. When the sheet is held straight little volume is heard. However, when it is bent slightly it, the sheet becomes a reasonably efficient speaker. FIG. 18C shows the concept of joining a curved or domed PVDF sheet 1820 to a microneedle 1830. The curved sheet 1820 of PVDF acts as a diaphragmatic pump. Prior to eating, the pump is activated to deliver a bolus of GLP-1 or other regulating hormone. In other examples, unimorph plates developed by NASA are used where these unimorph plates develop a stress on one side and after processing the stress deforms the plate into a curved plate. Similarly, an iontophoretic transdermal drug delivery system such as the Ionsys system available from ALZA, an affiliate of Johnson and Johnson of New Brunswick, N.J. may be employed.

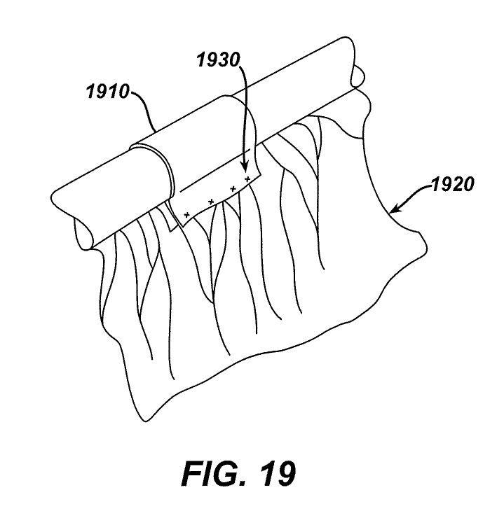

In an alternative embodiment seen in FIG. 19, since the mesentery 1920 supplying the bowel tends to interfere with a full circumferential engagement of the bowel, a spring loaded c-shaped configuration that encompasses the vast majority of the circumference of the bowel is positioned. A patch 1910 is stitched, t-tagged, or otherwise fastened together at various points 1930 through the portion of mesentery 1920. This provides the advantage of delivery of a therapeutic substance through the bowel lumen without transection. In turn, the chance of leaking and infection is greatly reduced.

In yet another embodiment, a delivery interface uses a transluminal needle. The needle is used to puncture the lumen during installation of the therapeutic substance delivery system. A fixturing means (e.g. suture) is then used to restrict dislodging of the needle. The needle may have a surface texture that promotes tissue adhesion and further secures the needle.

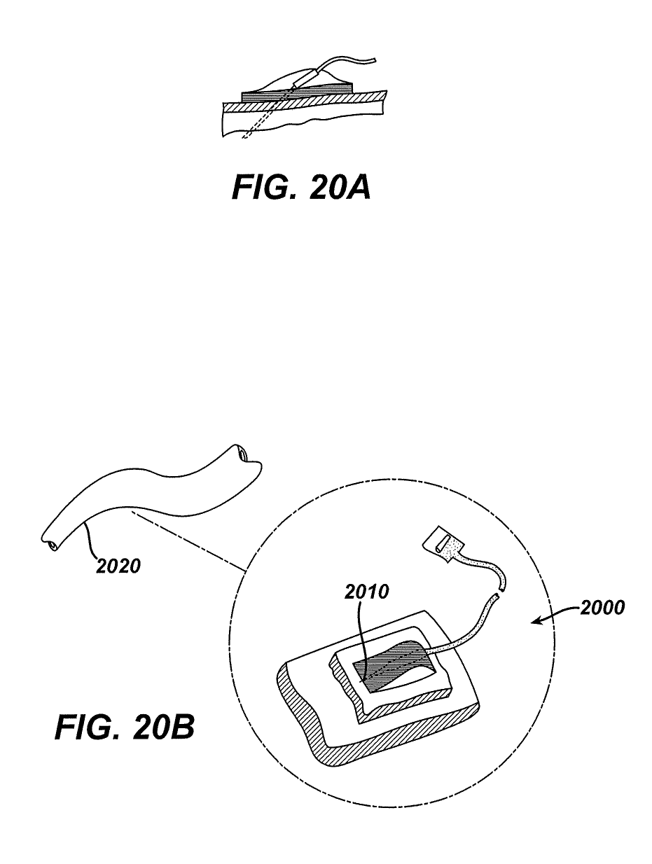

In still another embodiment, as shown in FIGS. 20A and 20B, a delivery interface 2000 uses a needle 2010 in fluid connection to the transfer system and connected to a mechanism that periodically and briefly punctures the needle 2010 through the lumen of the intestine 2020 at the deliver site and triggers the exit of therapeutic substance. After a periodic and brief puncture, the needle 2010 stops the delivery of substance and retracts back through the original hole. The size of the needle 2010 and infrequency of the punctures permits the lumen 2020 to heal between punctures. The needle 2010 may be advanced through the wall of the lumen 2020 hydraulically. After the needle 2010 has extended to its fullest extent, the therapeutic is expelled. The therapeutic may serve as a hydraulic fluid. Since the puncture force of the lumen wall is very low and the needle 2010 has a small diameter with a small opening (about 0.01-0.1 inches), the needle 2010 would pierce the tissue at a lower pressure than required to expel the fluid into the lumen 2020. On the proximal end, a motivating force may be an inductively coupled electromagnet subcutaneously positioned and activated by a matching coil in a hand-held unit. Each time the patient requires a delivery of therapeutic substances, the hand-held unit is moved in close proximity to the port to cause activation of the delivery system.

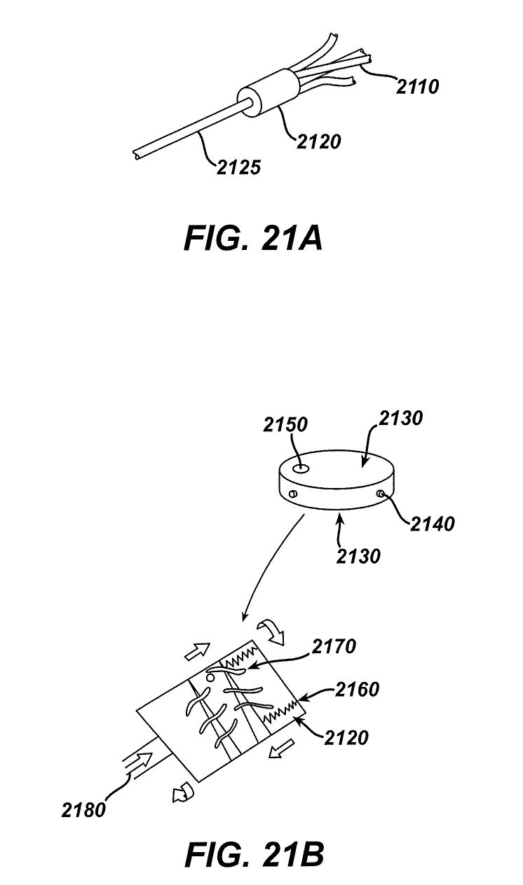

In yet another embodiment as seen with respect to FIGS. 21A and 21B, a number of needles extending from lines 2110 on an indexing chamber 2120 connected to a supply line 2125 are activated, such that any needle only punctures the lumen periodically, allowing each portion of the lumen wall to heal before the next puncture. Alternating of needles occurs in some examples by means of the indexing chamber 2120 which responds to pressure increases by rotating an indexing disk 2130 successively to each of a plurality of exit catheters. The indexing disk 2130 is forced distally by the pressure rise. Helical protrusions in the chamber 2120 inner wall engage with bosses 2140 on the disk 2130, forcing rotation by a set increment. As the disk 2130 bottoms out, the off-center hole 2150 on the disk 2130 aligns with one of the outlet catheter holes. A needle receives the pressure to activate it. When the pressure is reduced, return springs 2160 in the chamber push the disk 2130 proximally, causing the disk 2130 to engage with a second set of helical protrusions 2170 which index the disk further. At this point, it engages the next set of helical protrusions 2170 such that it aligns for pressure supply inflow 2180 with the next hole on the next pressure cycle.

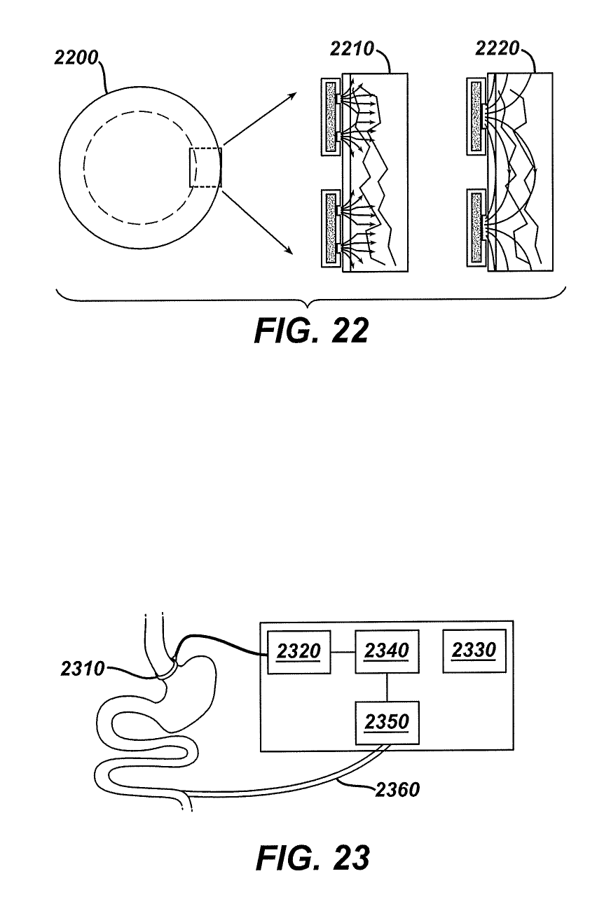

Turning now to FIGS. 22-25, electrical stimulation of the L-cells in the ileum lowers the threshold needed to release hormones such as GLP-1 into the system. Thus, combining an electrical stimulation system with the therapeutic substance system via a stent electrode 2200 having monopolar 2210 or bipolar 2220 centers may provide a more powerful and desirable therapeutic effect. In one embodiment, the electrodes are part of the same physical structure as the therapeutic substance delivery interface. Configuring the design insures that the therapeutic substances are delivered to cells that have been stimulated. By doing so, less therapeutic substance will be required to stimulate the intestinal brake, reducing potentially invasive refills.

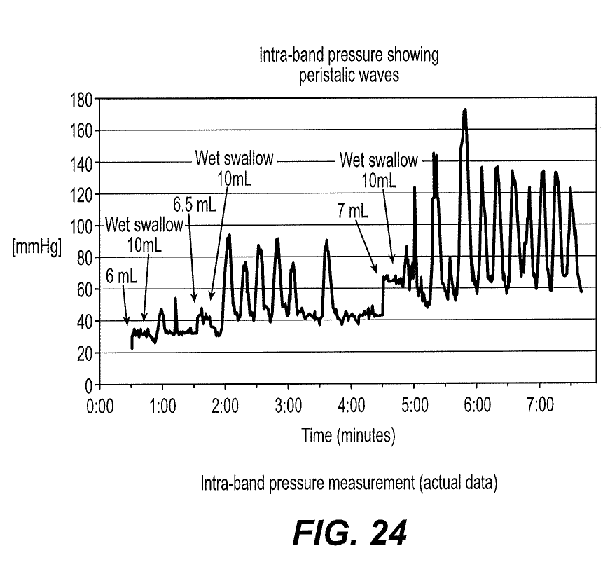

As seen with respect to FIG. 23, the implanted system may include an adjustable gastric band 2310, a pressure sensor 2320, a battery 2330, a microcontroller 2340, a signal generator 2350, and electrical leads 2360 connecting to the ileum. As shown, the gastric band 2310 is used as a reservoir with the therapeutic substance. A delivery catheter connects the reservoir fluidly with the delivery interface. The catheter also has electric leads for the delivery of electrical stimulation pulses. Recognition of waves within pressure data may occur via time-averaged peak-detection algorithms wherein average baseline pressures are compared over periods of time or via machine learning algorithms/neural nets. As seen in FIG. 24, intra-gastric pressure from peristaltic waves is typically known as a function of time after ingestion of food. Such pressure data is used to optimize the machine learning algorithms.

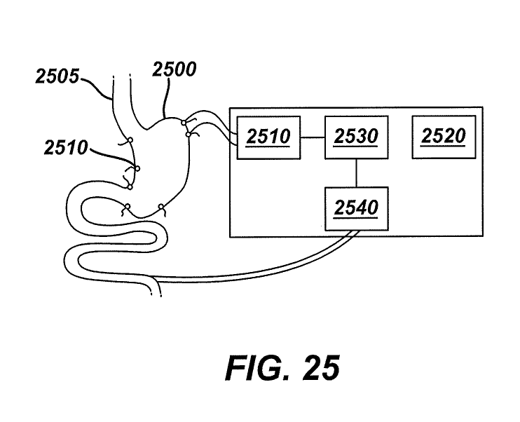

In another embodiment of FIG. 25, peristalsis is recognized by piezo ceramic transducers implanted into the wall of the stomach 2500 or esophagus 2505. The detection of peristalsis by a pressure sensor 2510 operatively coupled to a battery 2520 and control system 2530 then triggers electrical stimulation of the ileum by means of a signal generator 2540.

Several benefits are provided by the embodiments of the present invention. Gastric banding is already a broadly accepted treatment for obesity and greater weight loss control can be achieved by the embodiments of the present invention than with gastric banding alone. In addition, there is a greater likelihood of success for a larger percentage of patients because it utilizes more than one mechanism for weight loss and combines them synergistically. Further, the embodiments presented are reversible. Intra-band pressure data is used to improve control of the intestinal brake device.

The combination of therapeutic substance with stimulation multiplies the effect of the intestinal brake. A delivery interface utilizes stimulation to increase the effect of the introduced therapeutic. The invention may stimulate the segmentation (motility) process in the ileum through mechanical, electrical, and/or chemical means and also can be externally controlled or adjusted via a wireless connection. The present invention requires surgical placement yet is expected to be relatively easy in intervention similar to use of a gastric band. No surgical cutting of tissue is anticipated in the gastrointestinal tract.

In a first example using mechanical stimulation, a series of bands is placed around the ileum at appropriate distances from each other. The bands are in electrical communication with each other to allow system control. The bands have relaxed and contracted states that are adjustable. The mechanism of contraction and relaxation may be regulated by a variety of methods including use of a hose clamp worm gear driven by a subminiature motor, pressurization of saline by a subminiature pump filling and emptying to an overflow reservoir, and also by means of electroactive polymer-based segments, as examples and not by way of limitation.

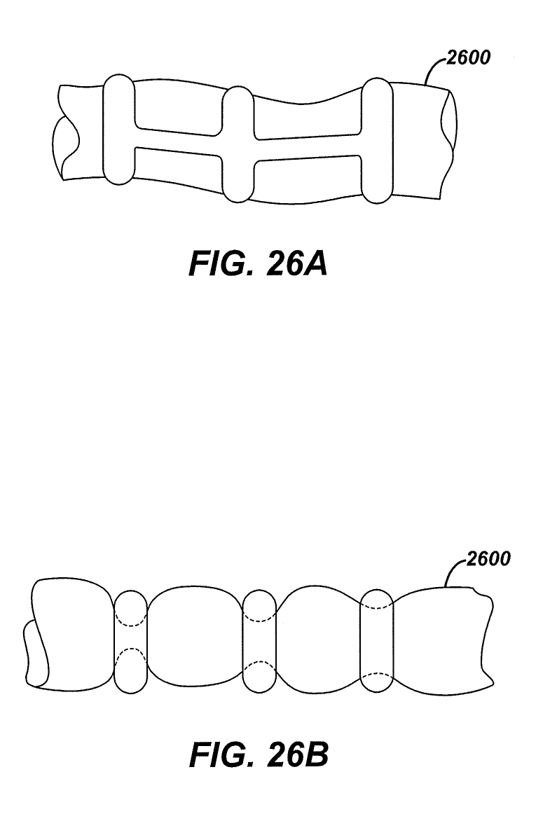

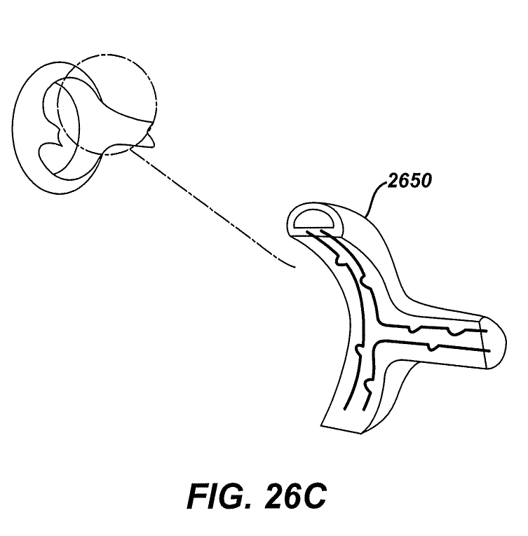

Once placed, these bands are synchronized to work together at the appropriate frequency. A MEMs pressure sensor similar to what is currently used in tires may monitor interactions with the ileum and with wireless control and the right algorithm may stimulate hormones or GLP-1 without the presence of food. In another embodiment seen in FIGS. 26A and 26B, the mechanical device surrounding the bowel would be a gastric band 2600. A system in fluidic connection to the band would move fluid in and out of the band, causing expansion and contraction, and stimulate the bowel. In another embodiment as seen in FIG. 26C, the device uses electrical probes 2650 at the intestine/device interface. This provides electrical stimulation to the ileum neural system to overcome the signal threshold for segmentation to begin. And in yet another embodiment, the device supplies a chemical, drug, or hormonal therapy. The therapeutic substance is stored in the device reservoirs and refilled through a port similar to the gastric band.

In another embodiment, by raising the temperature in and around the vicinity of the ileum and jejunum, leptin is produced, hormones such as GLP-1 are released, and appetite suppression occurs. Increased temperatures may cause systemic inflammations such as fever or anorexia. By neutralizing circulating leptin, fever and anorexia are significantly moderated when inflammation occurs by means of bacterial endotoxin lipopolysaccharide, a pyrogen.

Any number of means may be used to deliver energy from an extracorporal source to be received by a device and turned to into heat. Radiofrequency or microwave antennas may be used to beam energy into the system. An objective is to limit the temperature rise to less than about one to two degrees Celsius. This implies on-board regulation on the receiver/heater or feedback with the external source. On board regulation may be based on a PTC material (ceramic or polymer) that limits current flow to the heater at a desired temperature. Simple switching from a bimetallic-like switch may be employed.

Polymer-based bands for use in a gastric banding are known in the art. In one example of a therapeutic delivery system, a band is wrapped around the esophagogastric junction to create a small pouch and a restriction. After placement, the band is filled with saline creating a pressurized collar. A filling port is connected to the band and is placed immediately under the skin. The physician can regulate fluid level in the band via the port. The same band technology can be used as a reservoir for GLP-1 or hormonal analogs and other incretins, nutrients, or substances. As used herein, the term “analogue” means a polypeptide which is a derivative of GLP-1, which derivative comprises addition, deletion, substitution of one or more amino acids, such that the polypeptide retains substantially the same function as native GLP-1. The saline may be replaced with these metabolic components properly maintained in a solution.

In this example, the band dumps the appropriate GLP-1 or hormonal analog when eating starts. Alternatively, the band may utilize a signal when eating starts such as via a pump/or squeezing action. The eating signal is tied to the individual hitting a switch that is integral to the port. The signal may be initiated by simply pressing on the skin in the vicinity of the band near an activating switch (e.g. an internal squeeze bulb). Other squeezing mechanisms may be the use of a coil that receives radiofrequency (RF) energy to create a magnetic field. Resultant magnetic forces in turn squeeze the volume of the band.

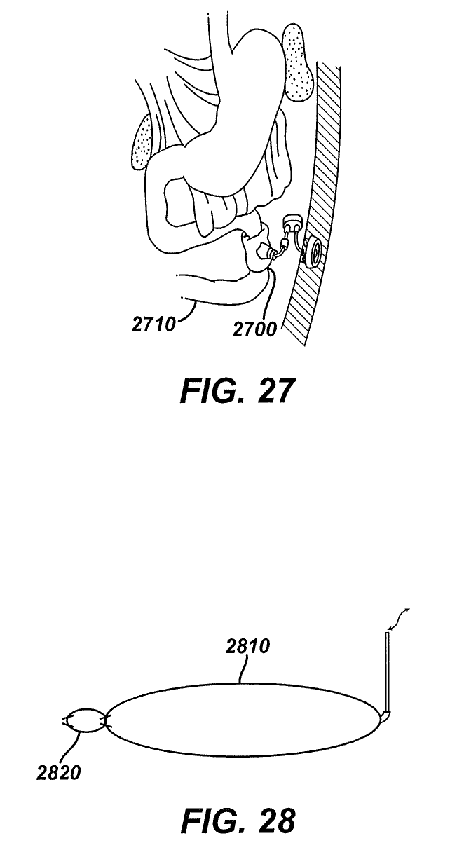

As seen in FIG. 27, the band 2700 is wrapped around a portion of the intestine 2710. The band 2700 or installed system may also include an exit port to release GLP-1 or other hormones. The wrapped band 2700 may include a nanomembrane that permits the GLP-1/hormone to be delivered systemically and in some examples picked up directly in the blood supply surrounding the intestine. The membrane may allow the GLP-1/hormone to leach out and have a steadystate low-level of GLP-1 to induce a feeling of satiety. Alternatively, GLP-1/hormone may be unloaded into the area around the band 2700.

In a second example, the band reservoir uses two chambers as seen in FIG. 28. A first chamber 2810 is a larger reservoir and a second chamber 2820 is a much smaller dosing volume. The dosing volume chamber 2820 may include check valves (e.g., duck bill valves) on its entrance and exit. When the dosing volume is squeezed, the exit valve opens and the entrance valve closes to introduce the dose into the tissue. The valve on the entrance side leaches such that the dosing volume chamber 2820 is slowly refilled by the reservoir volume chamber 2810. The dosing volume chamber 2820 may take the form of a small section of tubing. The internal volume of the tube would maintain the dose. The system as described may be manually operated, semi-automatically controlled, or fully automated.

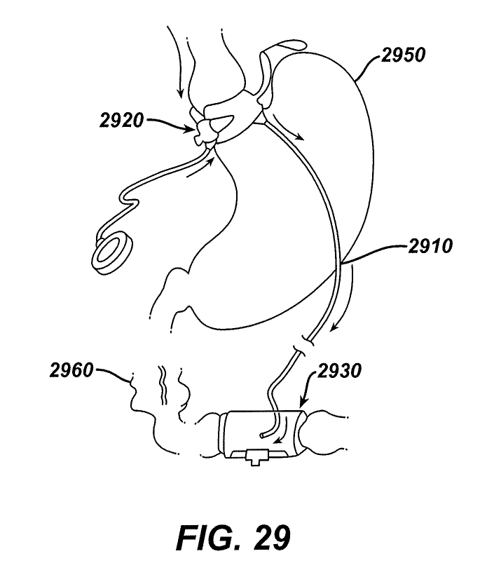

In a third example shown with respect to FIG. 29, a device employs existing banding technologies in a two-fold effort to regulate satiation. This device has two parts joined by a catheter 2910 and employs a series of one-way valves directing fluid flow downwards from first modified gastric band 2920 positioned above a fundus 2950 of a stomach toward a second sleeve 2930 positioned around a portion of a colon 2960. The first band 2920 may be a stretchable tube-like balloon that is volume-controlled by infusion with an inert liquid. The band 2920 may be modified such that there are two fluid filled compartments, one circumscribed within the next. One compartment may be connected to an external fill port. The other compartment may be filled with an active chemical or serum of calories and connected via catheter to the lower portion.

The first band 2920 of the invention provides physical restriction of the stomach as seen in FIG. 29. Upon consumption of food, increased pressure on the stomach imparts a physical stretch on the upper band 2920. That pressure redistributes fluid maintained in this upper band 2920 downward into the catheter 2910 through a series of one-way valves and into the second, lower sleeve 2930 that is situated around the ileal portion of the bowel. When enough pressure is exerted on the upper band 2920, displaced liquid from the band 2930 is released onto the ileum. This serum acts as the second component of the invention. Chemical signalling for satiation through influencing the ileum to impart the intestinal brake by way of a slow calorie release is achieved.

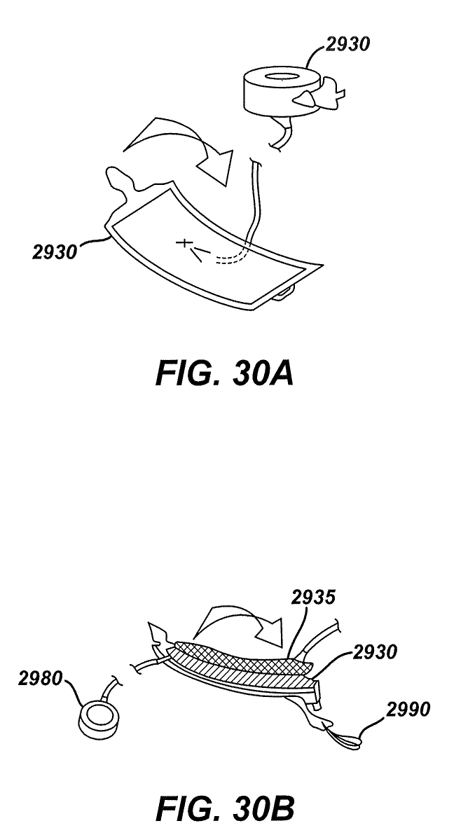

In FIGS. 30A and 30B, the sleeve 2930 may be a folded band comprised of a semi-permeable membrane 2935 that facilitates fluid transfer out of the device lumen and onto its envaginated contents. The sleeve 2930 may also be terminated by an electrical probe or stimulator that elicits a potential change in the ileum. The sleeve 2930 may be operatively coupled to a fill port 2980 and includes sutures 2990 or suturing equivalents to secure placement of the sleeve 2930.

In another example of the two-fold banding method, an agent is released that electrically influences the ileal portion of the bowl by altering the pH. In yet another variant example, a second restrictive band acts at a point along the alimentary system post pyloric sphincter. The band acts according to the same principles of the sleeve wherein pressure on the upper band forces fluid into the lower band and thus restricts food passage at the point of the lower band. This acts to slow down the emptying of the bowel and promotes satiety by prolonging the intestinal brake. The aforementioned embodiments of the present invention are generally minimally invasive, reversible, and may be implemented laparoscopically.

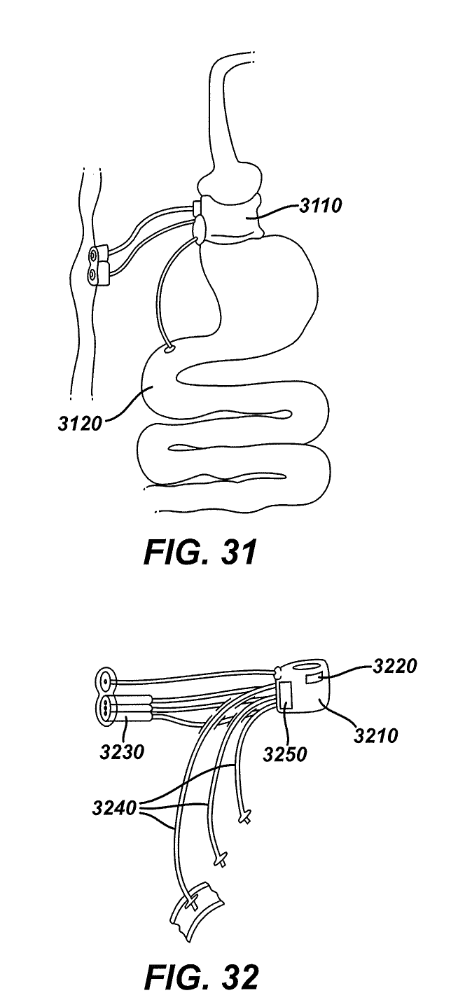

In yet another example seen with respect to FIG. 31, a gastric band reservoir includes a control system. A patient is fitted with an apparatus swallows a bolus of food. The bolus passes through the reduced aperture provided by a modified gastric band 3110. At that moment the gastric band 3110 experiences a localized increase in pressure. This increase in localized pressure triggers introduction of fluid into an intestinal tract 3120 of the patient. This introduction occurs in specific areas of the intestinal tract 3120, for instance the ileum, jejunum and/or the duodenum. This fluid may be a concentration of lipids or hormones to trigger satiety. One of the benefits of such a device is a significant decrease in the time required for satiety. The general concept is to provide the patient with stretch receptor feedback in the stomach and to initiate a hormonal cascade in the down stream gut in response. This represents a relatively simple alternative to the implementation of more drastic procedures.

In an electro-mechanical example seen with respect to FIG. 32, the band 3210 is equipped with one or more pressure sensors 3220 that monitor food intake by sensing the local increase of pressure during the passage of a food bolus. The device contains liquid reservoirs 3230 and at least one lumen 3240 in the form of a tube extending from the reservoir portion to a more distal portion of the bowel; duodenum, ileum, jejunum and/or the colon. The device also contains a pump 3250 in order to move the liquid/substance from the reservoir 3230 to the specific portion of the gastrointestinal tract. The liquid infused into one or more portions of the bowel may include specific amino acids, carbohydrates, glucose, lipids and/or hormones as examples. An infusion of one or more of these liquids in one or more gastrointestinal tract areas causes an effect of hormones released into the bloodstream which in turn causes the brain to slow down gastric motility or initiate the intestinal brake system within the human body. When this happens, the body slows down the digestion of food which allows the individual to feel full longer and create the sensation of satiety. Usually, the time between the initial food intake and the release of the hormones is a longer period of time than what would be optimal for satiation and satiety. The device infuses the nutrient/liquid immediately after sensing food intake creating a simultaneous reaction of satiation and initial satiety working together to create optimal eating habits in which the individual that has this device surgically implanted would eat less and/or have a longer feeling of satiety between meals. The reservoirs are refillable with a variety of available liquids/nutrients.

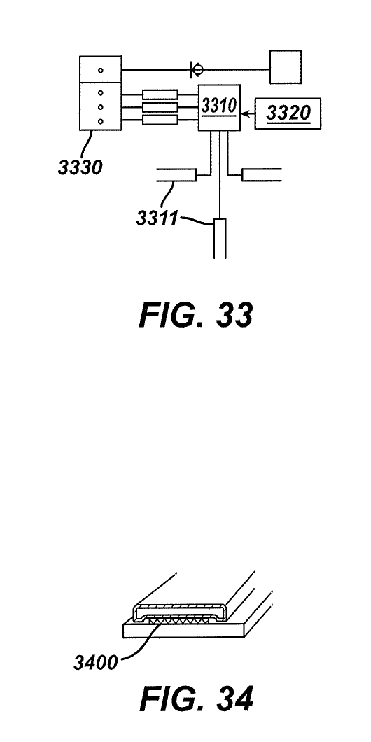

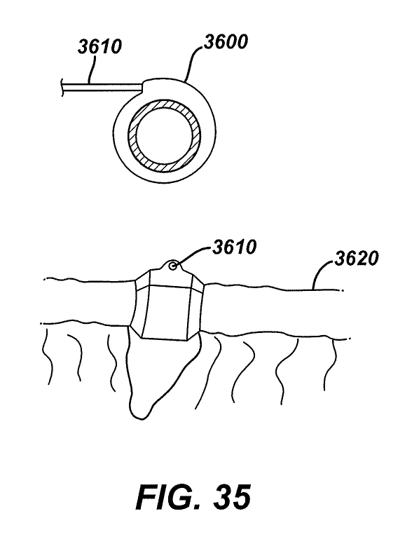

In other modifications that may be applied generally to the aforementioned embodiments and shown with respect to FIGS. 33-36, a number of additions and/or improvements may be incorporated. In a first example seen in FIG. 33, a pump 3310 is used to supply inlets to multiple ports 3311 of the small intestine. The system may include a pressure sensor 3320 and a control system 3330. In another example seen in FIG. 34, a piezo film 3400 is added to a band to recharge an electromechanical system such that when peristalsis occurs, recharging is triggered. Induction principles may be included in the recharging system. Referring back to FIGS. 5A and 5B and associated description, a mechanical approach is shown utilizing a pumping section composed of valves. The valves may comprise dielectrics and may be nested between the more elastic portion of the band/fluid reservoir and the outer less elastic portion of the band used for holding the band together. In FIG. 35, the cuff concept provides an alternative to a direct line introduction, such as to the small intestine. Due to complications associated with stomal creation, most direct conduit lines into a lumen can only be tolerated for a couple weeks at the most. Therefore a cuff 3600 is created having a fluid conduit 3610 and the cuff 3600 is positioned around a portion of the small intestine 3620 creating a band like device that envelopes a particular region of the small intestine 3620. The region between the intestinal band and the intestine is where the drug/hormone is introduced. The drug crosses the serosol layer and is introduced through the drug stream locally.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

I/We claim:

1. A method of delivering a therapeutic substance to achieve intestinal brake in a patient, the method comprising the steps of:
a. providing a transfer system, the transfer system comprising
i. a reservoir for receiving and/or containing at least one therapeutic substance and comprising a delivery interface; and
ii. a catheter, wherein said catheter is operably connected to said reservoir;
b. placing said catheter intraluminally into a small bowel of said patient; and
c. administering said at least one therapeutic substance to a gastrointestinal tract of said patient via said catheter, wherein said act of administering said therapeutic substance results in intestinal brake in said patient.

2. The method of claim 1 wherein said transfer system comprises a harvested portion of the body of the patient, the harvested portion comprising L-cells selected from a saphenous vein, a diameter reduced portion of the gastrointestinal tract left attached to the mesentery, or a combination thereof, wherein said harvested portion receives transfer of therapeutic agents via said catheter.

3. The method of claim 1, wherein said reservoir for receiving and/or containing at least one therapeutic substance further comprises a head loss coil,
wherein said head loss coil connects said reservoir for receiving and/or containing at least one therapeutic substance with said delivery interface;
wherein said therapeutic substance is contained within said reservoir at a first pressure;
wherein said head loss coil permits said therapeutic substance to be administered to said patient at a second pressure;
wherein said first pressure is higher than said second pressure.

4. The method of claim 1 wherein said act of administering said therapeutic substance is facilitated by the effect of peristaltic motion of said gastrointestinal tract on said transfer system in said patient.

5. The method of claim 1 wherein said therapeutic substance is selected from at least one of GLP-1, a GLP-1 analog, or a combination thereof.

6. The method of claim 1 wherein said reservoir is selected from a gastric band, a water absorbent pill, or a combination thereof.

7. The method of claim 1 wherein said catheter comprises a one way valve.

8. The method of claim 1 wherein said catheter comprises a perforated tube.

9. The method of claim 1 wherein said catheter comprises an inline pump.

10. The method of claim 1 wherein said catheter comprises a plurality of pumps, wherein said plurality of pumps provides peristaltic pumping.

11. The method of claim 1 wherein said transfer system further comprises a feature selected from an adjustable gastric band, a pressure sensor, a battery, a microcontroller, a signal generator, and electrical leads, or a combination thererof.

12. The method of claim 1 wherein said reservoir is capable of being refilled via a nasal fill port.

13. The method of claim 1 wherein said delivery interface comprises a feature selected from at least one of a fine mesh capable of preventing particulate from entering said transfer system, a one way valve, a radiopaque material for location of the delivery interface, a perforated tube, a microneedle array, a piezoelectric polymer film sheet, a transluminal needle, or a combination thereof.

14. The method of claim 1 further comprising the step of delivering an electrical pulse to the gastrointestinal tract of said patient, wherein said electrical pulse enhances the intestinal brake effect of said method.

15. The method of claim 1 further comprising the steps of
a. detecting peristalsis in the gastrointestinal tract of said patient using a transducer implanted in an organ selected from stomach, esophagus, or a combination thereof, wherein said detecting step is carried out wirelessly; and
b. delivering an electrical pulse to the gastrointestinal tract of said patient, wherein said electrical pulse stimulates an ileum of said patient such that GLP-1 is released.

16. A method of delivering a therapeutic substance to achieve intestinal brake in a patient, the method comprising the steps of:
a. wrapping at least one band around a location selected from an esophagoastric junction, a stomach, an intestine, or a combination thereof, wherein the act of wrapping creates a small pouch and a restriction;
b. filling said at least one band with a fluid, wherein said act of filling creates a pressurized collar;
c. connecting a filling port to said at least one band immediately under the skin, sufficient to allow regulation of a fluid level within said band externally;
d. selecting a substance for delivery to said at least one band; and
e. delivering said substance to said at least one band;
wherein said at least one band delivers said substance to said patient, and wherein said substance is selected from at least one of GLP-1, a GLP-1 analog, incretins, nutrients, or a combination thereof.

17. The method of claim 16 wherein said at least one band is responsive to a signal, such that communication of the signal to said at least one band causes release of said substance from said at least one band into said patient when said patient begins eating.

18. The method of claim 16 wherein said band comprises a nanomembrane and releases said substance systemically at a level sufficient to induce a feeling of satiety.

19. The method of claim 16 wherein said method further comprises the step of situating a sleeve containing a fluid around an ileal portion of a bowel of said patient,
wherein the act of wrapping comprises wrapping at least one band around said stomach of said patient;
wherein said sleeve and said at least one band are operably connected; and
wherein an increased pressure in said at least one band results in a pressure redistribution in said sleeve sufficient to release said fluid from said sleeve into said ileal portion of said bowel.

20. A method of filling a reservoir implanted in a patient, the method comprising the steps of:
a. introducing a nasogastric tube comprising a feature selected from a magnetic ring, an elastomer fill, a stopper, an inflatable balloon, a bend, flexible arms, a spring, or a combination thereof, into a patient at a location that is not visible from outside said patient;
b. connecting said nasogastric tube with said reservoir;
c. anchoring said nasogastric tube within the nasal cavity;
d. accessing said nasogastric tube; and
e. introducing a substance into the reservoir through the nasogastric tube.
